(19) United States
(12) Patent Application Publication
(10) Pub. No.: US 2012/0052126 A1
(43) Pub. Date: Mar. 1, 2012

(54) Nanoemulsions Containing Antioxidants And Other Health-Promoting Compounds

(76) Inventors: Yashwant Pathak, Tampa, FL (US); Hieu T. Tran, LaGrange, KY (US)

(21) Appl. No.: 13/219,798

(22) Filed: Aug. 29, 2011

Related U.S. Application Data
(60) Provisional application No. 61/378,053, filed on Aug. 30, 2010.

(51) Int. Cl. A61K 9/14; A61K 31/385; A61K 31/122; A61K 31/198; A61P 3/02; A61K 31/59; A61K 31/51; A61K 31/525; A61K 31/375; A61P 39/06; A61K 31/355; A61K 31/05

(52) U.S. Cl. 424/490; 514/458; 514/440; 424/94.1; 514/562; 514/733; 514/167; 514/168; 514/276; 514/251; 514/474

(57) ABSTRACT

Nanoemulsions contain particles that comprise an outer shell layer containing at least one oil and a core portion containing at least one antioxidant and, optionally, other health-promoting compounds, wherein the nanoemulsions are relatively stable for prolonged periods without significant change in physical properties and are suitable for administering to humans and other mammals orally, topically, intravenously, transdermally, and subcutaneously.

### CROSS REFERENCE TO RELATED U.S. APPLICATION

This application claims priority to U.S. Provisional Application No. 61/378,053 filed on Aug. 30, 2010.

### BACKGROUND

The health-promoting features of antioxidants include the protection of human cells, and other mammalian cells, from the injurious or cytotoxic effects of reactive chemical species, which include but are not limited to free radicals. Free radicals form when an atom or molecule has at least one unpaired electron, making the atom or molecule unstable and highly reactive. In that state, the tendency of an atom or molecule to attract electrons from other atoms or molecules is increased. For example, an oxygen molecule (O₂) having at least one unpaired electron is a type of free radical. Although the formation of reactive chemical species within or around human and other mammalian cells is a natural process, some environmental factors are known to increase the rate of their formation. Reactive chemical species are associated with other molecules besides oxygen, such as, for example, hydrogen peroxide, hydroxyl radical, hypochlorous acid, nitrous oxide, singlet oxygen, and super oxide radical.

The negative effects of reactive chemical species include, but are not limited to, damage to DNA, inflammation, mitochondrial abnormalities, and other physiologic disturbances. Because of their unstable and highly reactive state, reactive chemical species react with a number of molecules, such as, for example, lipid, protein, and carbohydrate molecules; RNA molecules; and DNA molecules. These reactions alter the structure and function of the molecules. The effects of such alterations cause injury to cells and tissues by interfering with cell membranes, as well as important functions of the cells, including, but not limited to, those involved in cell-to-cell communication, protein synthesis, transcription, translation, electron transport, mitochondrial function, the passage of neurotransmitters across synapses, and the neuroprotective functions of myelin.

Such injuries cause, or contribute to, sometimes irreversibly, many diseases and unhealthy states. These include, but are not limited to, Alzheimer's disease, dementia, Parkinson's disease, cardiovascular disease, cancer, atherosclerosis, rheumatoid arthritis, and impaired vision. Over time, the formation of reactive chemical species is associated with aging. Conversely, the ability to limit the formation and/or effects of reactive chemical species promotes health and longevity. The descriptions and teachings disclosed herein include, but are not limited to, those related to conditions, diseases, and states affecting the nervous system, such as, for example, brain tissue.

Many antioxidants are known to limit the formation and/or effects of reactive chemical species. These include, but are not limited to, vitamin A, vitamin E, beta carotene, curcumin, Coenzyme Q10, ethoxyquin, ginkgo biloba, glutathione, huperzine, lipoic acid, lycopene, melatonin, quinones, resveratrol, quercetin, catechin, epicatechin, hydroxytyrosol, 5-hydroxytryptophan, squalene, apolipoprotein E, bilirubin, creatine, uric acid, carotenoids, flavonoids, polyphenolic compounds, in addition to various constituents of fruits, vegetables and legumes, such as, for example, lignans. Some antioxidants are natural compounds, while various synthetic and semi-synthetic compounds are also antioxidants.

Accordingly, the embodiments described and taught herein are nanoemulsions containing antioxidants and/or other health-promoting compounds. Other health-promoting compounds include, but are not limited to, proteins derived from herbal and natural sources, such as, for example, soy protein and whey protein.

In an embodiment, a nanoemulsion is formed that contains an antioxidant, in which a comparatively high proportion of the contents is, following consumption by a human, or other mammal, processed in a manner that delivers the antioxidant to cells in the brain, such as, for example neurons. In other embodiments, the nanoemulsions contain antioxidants and other health-promoting compounds, in which a comparatively high proportion of the contents is, following consumption by a human, or other mammal, processed in a manner that delivers the antioxidants and other health-promoting compounds to cells in the brain, such as, for example, neurons. Delivery of the antioxidants and other health-promoting compounds to cells in the brain requires that they pass the blood brain barrier.

In addition to the blood brain barrier, other systems and physiological mechanisms occur naturally in humans, and other mammals, which prevent some portion of antioxidants and other health-promoting compounds from reaching the cells of the brain. Such other systems and physiological mechanisms include, but are not limited to, metabolism by the hepatic system, absorption into cells located away from the brain, metabolism in the blood or non-brain tissues, and elimination by the renal system. According to the descriptions and teachings of the embodiments disclosed herein, a portion of antioxidants and/or other health-promoting compounds consumed, by a human or other mammal, is processed in a manner that bypasses such other systems and physiological mechanisms, or is otherwise marked by a degree of avoidance of such other systems and physiological mechanisms.

One way to increase the quantity of antioxidant, or other health-promoting compounds, that reaches the cells of the brain is to consume them in larger quantities. However, several disadvantages accompany the consumption of large quantities. One disadvantage is that absorption is dose dependent. In general, the greater the dose, the slower the rate of absorption, which leads to reduced bioavailability. In some cases, but not all cases, a sustained release form of antioxidant or other health-promoting compound is available, which provides greater bioavailability compared to equivalent pure doses. Notwithstanding, in general, absorption becomes less efficient as the dose is increased.

Another disadvantage, associated with consuming larger quantities of antioxidants, or other health-promoting compounds, is that the antioxidants or other health-promoting compounds are usually suspended or dissolved in liquid, or otherwise must be consumed by mouth as part of some liquid or semi-liquid matter. Generally, therefore, to increase the quantity of antioxidants or other health-promoting compounds consumed, a larger volume of liquid or semi-liquid matter must be consumed. However, some individuals cannot tolerate the larger volumes, or can consume such volumes only with difficulty. Patients who suffer from dysphasia, trauma, or burns, for example, in addition to the elderly or patients with dementia, are some of the kinds of individuals who would face difficulty or exacerbation of their problems, if they were to attempt to consume larger volumes of liquid or semi-liquid matter.

Accordingly, nanoemulsions containing antioxidants, and/or other health-promoting compounds, provide an advantage because the nanoemulsions can be consumed at relatively low volumes. This tends to reduce the undesired effects associated with consuming large volumes, yet still provides for the delivery of a desirable amount of those antioxidants, and/or health-promoting compounds, to the brain cells of humans or mammals. In some embodiments, these are suitable for use as nutraceuticals and/or in combination with traditional therapies (not claimed herein) for neurodegenerative and other diseases affecting cognition and mental processing.

### BRIEF DESCRIPTION OF FIGURES

FIG. 1 is an image from a scanning electron microscope showing oil globules in which at least one antioxidant, and/or other health-promoting compound(s), is incorporated, according to an embodiment taught and described herein.

FIG. 2 is another image from a scanning electron microscope showing oil globules in which at least one antioxidant, and/or other health-promoting compound(s), is incorporated, according to an embodiment taught and described herein.

FIG. 3 is a graph showing comparative levels of malonaldehyde production in mice following oral administration of nanoemulsions containing antioxidants as further discussed in Example 7.

### MULTIPLE EMBODIMENTS AND ALTERNATIVES

In an embodiment, an emulsion is prepared that includes an oil phase, and a water phase. The oil phase includes oil, an emulsifying agent, and one or more antioxidants. The water phase includes water. Optionally, in some embodiments, the water phase includes a preservative, and a stabilizing agent.

In some embodiments, the oil is unsaturated oil, such as, for example, soybean oil, olive oil, safflower oil, canola oil, or a combination of two or more of those. Alternatively, the oil is an antioxidant, such as, for example, vitamin E or vitamin A.

In general, the emulsifying agent is a phospholipid, a natural emulsifying agent from a vegetable source, a natural emulsifying agent from animal source, a polymeric emulsifying agent, a semi-synthetic polysaccharide, a synthetic emulsifying agent, an anionic surfactant, a cationic surfactant, a non-ionic surfactant, a finely divided solid, or a combination of two or more of the above. In some embodiments, the emulsifying agent is one of, or a combination of, those chosen from the group tragacanth, acacia, agar, chondrus, pectin, lecithin, lanolin, cholesterol, methyl cellulose, sodium carboxylmethyl cellulose, hydroxylpropyl cellulose, cetrimide, benzalkonium chloride, benzethonium chloride, glyceryl monostearate, propylene glycol monosteatrate, polyoxyl stearate, bentonite, aluminium magnesium stearate, attapulgite, colloidal anhydrous silica, and hectorite. The agents described and taught herein, which are used as emulsifying agents, preservatives, and stabilizers, are available for purchase from a number of commercial sources.

In some embodiments, the antioxidant is a natural compound. Alternatively, the antioxidant is a synthetic or semi-synthetic compound. In some embodiments, the antioxidant of the oil phase is chosen from the group vitamin A, vitamin E, beta carotene, curcumin, Coenzyme Q10, ethoxyquin, ginkgo biloba, glutathione, huperzine, lipoic acid, lycopene, melatonin, quinones, resveratrol, quercetin, catechin, epicatechin, hydroxytyrosol, 5-hydroxytryptophan, apolipoprotein E, bilirubin, creatine, uric acid, carotenoids, flavonoids, and polyphenolic compounds. Alternatively, two or more of those are combined as antioxidants.

Optionally, other health-promoting compounds are added to the oil phase, such as, for example, soy protein, whey protein, vitamin A, vitamin D, and/or other oil soluble vitamins, either individually, or in combination.

The emulsifying agent, the antioxidant(s), and the other oil soluble ingredients that are added to the oil phase, if any, are stirred for an adequate amount of time ranging from about 15 minutes to about 60 minutes, until they are substantially dissolved in the oil, such that a substantially clear solution is obtained. However, in some embodiments, depending on the selection of antioxidant(s), and/or other health-promoting compound(s), stirring for longer durations up to 8 hours is performed. Generally, the stirring time is proportional to the size of the batch. Stirring is accomplished by any one or more of various ways, such as are known to persons possessing ordinary skill in the art.

For the water phase, water is obtained, prepared and/or purified, by any one or more of various ways, such as are known to persons possessing ordinary skill in the art. Such ways may include, but are not limited to, the use of ion exchange columns with a suitable resin as selectably chosen by a person of ordinary skill. In some embodiments, the preservative is from the parabens family or sulfite family, or derivatives thereof. In some embodiments, the preservative of the water phase is chosen from the group methylparaben, propylparaben, sodium metabisulphite, or a combination of those. In some embodiments, the stabilizing agent of the water phase is chosen from the group ethylene glycol, glycerin, liquid PEG polymers, polyethylene glycol, propylene glycol, other glycols, or two or more of those in combination, including commercially sold embodiments of these agents, such as polyethylene glycol (PEG) 400 and Pluronic® F68. Optionally, glycerin is used to stabilize and to promote viscosity. In some embodiments, Pluronic® F68 is used as a stabilizer and for its complementary emulsifying effects.

In some embodiments, squalene, is added to the water phase as a water soluble antioxidant. Alternatively, aspartame is added to the water phase. Optionally, the two water soluble antioxidants are added in combination. In some embodiments, one or more water soluble vitamins, such as, for example, vitamin C (e.g., ascorbic acid), B vitamins (eg., vitamin B₁, vitamin B₆, vitamin B₁₂) and/or other nutritional components as may be obtained commercially, are added to the water phase as other health-promoting compounds.

In some embodiments, the preservative, stabilizing agent, and other ingredients as described herein, if any, are added to the water, and stirred until the water soluble ingredients are substantially dissolved in the water, such that a substantially clear solution is obtained. In some embodiments, each ingredient is added separately to a distinct volume of water, stirred until substantially clear, and then the separate solutions are mixed. Stirring is performed by any one or more of various ways, such as are known to persons possessing ordinary skill in the art. In some embodiments, stirring of the water phase occurs for about 10 minutes to about 60 minutes.

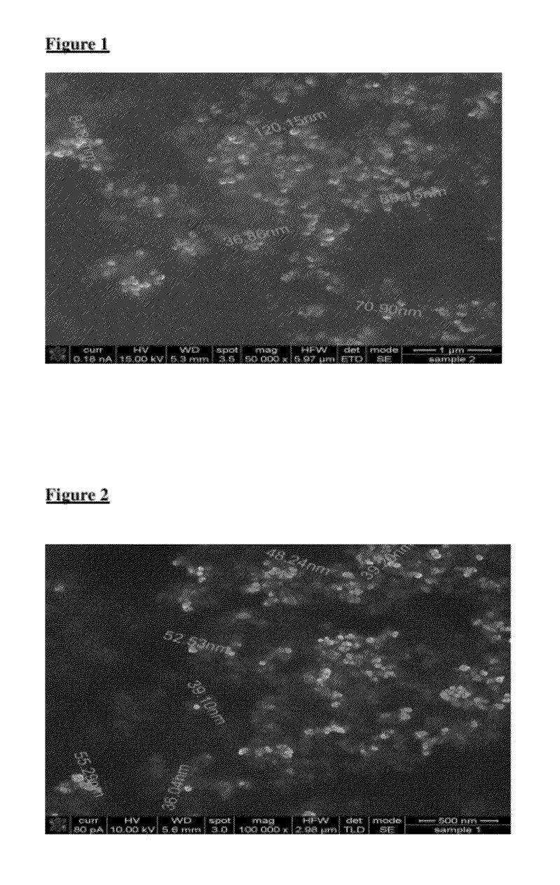

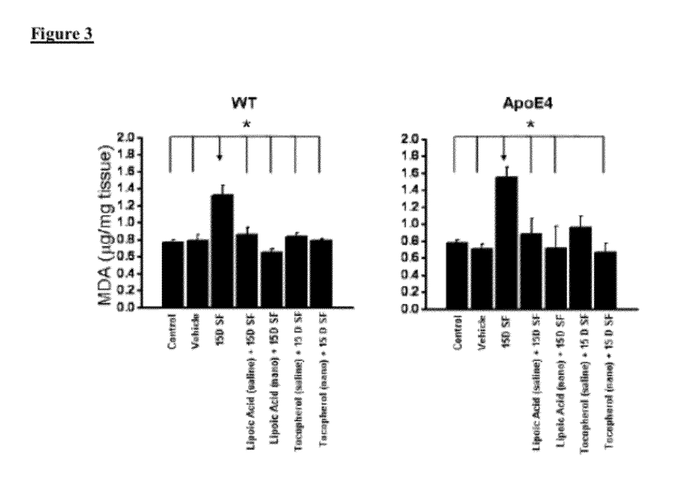

After preparing the oil phase and the water phase, as described above, the nanoemulsion is formed by dispersing the oil phase into the water phase. During this step, the oil forms into globules ranging in diameter from about 36 nanometers (nm) to about 500 nm. In some embodiments, the oil globules range in diameter from about 50 nm to about 350 nm. The images contained in FIG. 1 and FIG. 2 show the size of the oil globules as being within these ranges. Preferably, mean particle size distribution of the globules is about 180 nm to about 200 nm in diameter. The antioxidants, and other health-promoting compounds, are incorporated within the oil globules as the dispersing of the oil phase into the water phase occurs.

The emulsifying agent provides an outer layer that surrounds each oil globule. Intermolecular repelling forces (positive-to-positive electrical charge) between and among the oil globules keep each one separate from other oil globules. These forces limit coalescence, settling, creaming, and other undesired effects between or among the individual oil globules. The emulsifying agent supports the oil globules, and enables them to remain substantially intact and of consistent size over time during processing and storage of the nanoemulsions, thus promoting stability. In some embodiments, the nanoemulsions are stored in containers that block or limit direct light exposure, e.g., brown glass laboratory bottles.

In some embodiments, in order to disperse the oil phase into the water phase, a high-speed homogenizer is used. Optionally, the dispersing of the oil phase into the water phase is accomplished using a Misonix sonicator. Dispersing time ranged from about 10 minutes to about 60 minutes. In some embodiments, the dispersion of the oil phase into the water phase occurs in a single interval. Alternatively, the dispersion occurs over two or more separate intervals, of approximately equal duration.

In some embodiments, whey protein, soy protein, oil soluble vitamins, and water soluble vitamins are included as other health-promoting compounds. The inclusion of these provides relatively low volume, relatively high calorie, nanoemulsions containing antioxidants and other health-promoting compounds.

In some embodiments, pH of the nanoemulsions ranges from about 3.5 to about 8.5 at approximately 20° C. In general, the viscosity of the nanoemulsions provides adequate stability and consistency while maintaining desired flow properties at room temperature.

After an individual consumes a nanoemulsion, containing antioxidants and/or other health-promoting compounds, as formed according to multiple alternative teachings and descriptions of embodiments herein, the lipophilic property of the oil globules, combined with their relatively small size, will increase the tendency of the oil globules to enter the lymphatic system. This would be similar to the way chylomicrons enter the lymphatic system. Chylomicrons are lipophilic bodies with particle size from about 500 nm to about 1,500 nm, which are normally taken up by the lymphatic system, thus bypassing the liver metabolism. Consequently, upon exiting the lymphatic system into the blood circulation, the oil globules, with the antioxidant(s) and other health-promoting compounds incorporated therein, will possess a greater tendency to avoid or bypass metabolism by the hepatic system, to avoid or bypass absorption into cells located away from the brain, to avoid or bypass metabolism in the blood or non-brain tissues, and to avoid or bypass elimination by the renal system, and other such systems and physiological mechanisms that tend to prevent or limit the proportion of antioxidant(s) and other health-promoting compounds from reaching the cells of the brain. Moreover, this will increase the proportion of antioxidant(s) and other health-promoting compounds delivered to the brain, in part, because the oil globules' lipophilicity enables them, and the antioxidants and/or other health-promoting compounds, to pass the blood brain barrier. In addition to oral, other methods of administration include oral administration, topical, intravenous, transdermal, and subcutaneous administration, all of which are known methods to those possessing ordinary skill in the art.

Various alternative nano-emulsion formulations are shown in the following examples:

### Base Formulations I-III

TABLE 1
Showing ingredients, by percentage weight, for Base
Formulations I-III(ascending order, from left to right,
of soybean oil content and lecithin content).

| | I | II | III |
|---|---|---|---|
| soybean oil | 20% | 25% | 30% |
| lecithin | 1.5% | 2.0% | 3.0% |
| glycerin | 2.5% | 2.5% | 2.5% |
| PEG 400 | 5.0% | 5.0% | 5.0% |
| purified water | 71% | 65.5% | 59.5% |
| Total: | 100% | 100% | 100% |

For each of the base formulations, a 10 gm formulation was prepared, as follows. Base formulation I: 2.0 gm of soybean oil, 0.15 gm of lecithin, 7.1 gm of purified water, 0.25 gm of glycerin, and 0.5 gm of PEG 400. Base formulation II: 2.5 gm of soybean oil, 0.2 gm of lecithin, 6.55 gm of purified water, 0.25 gm of glycerin, and 0.5 gm of PEG 400. Base formulation III: 3.0 gm of soybean oil, 0.3 gm of lecithin, 5.95 gm of purified water, 0.25 gm of glycerin, and 0.5 gm of PEG 400.

For each of these, the constituents of the oil phase were combined and then stirred. The constituents of the water phase were combined and then stirred. The oil phase was then dispersed in the water phase. The steps of adding, stirring, and dispersing were performed according to the various alternative descriptions and teachings set forth herein. For a 25 mL batch, stirring generally was performed for about 15 minutes. For a 5 L batch, stirring generally was performed for about one hour.

### Examples 1-6

### Sample Formulations with Antioxidants

Base formulation II was used for Examples 1-6. Other constituents were added, as shown in Table 2.

TABLE 2
Constituents for Examples 1-6.

| | % by weight | Quantity* | |
|---|---|---|---|
| Oil phase: | | | |
| soybean oil | 25% | 2.5 | mL |
| lecithin | 3% | 0.3 | gm |
| antioxidant* * | | | |
| Water phase: | | | |
| methylparaben | 1% | 0.1 | gm |
| proplyparaben | 1% | 0.1 | gm |
| Pluronic F ® 68 | 1% | 0.1 | gm |
| glycerin | 2.5% | 0.25 | mL |
| PEG 400 | 5% | 0.5 | mL |

| | | |
|---|---|---|
| purified water | (to 100%) | (to 10 mL) |

*per 10 mL batch
**Antioxidants were as follows. Example 1: none. Example 2: vitamin E (50 mg). Example 3: lipoic acid (100 mg). Example 4: CoEnzyme Q10 (100 mg). Example 5: glutathione (500 mg). Example 6: resveratrol (200 mg).

For each of Examples 1-6, 10 mL and 50 mL batches were prepared, according to the descriptions and teachings of the multiple alternative embodiments set forth herein. pH of Examples 1-6 ranged from about 3.5 to about 6.0. Viscosity of Examples 1-6 ranged from about 5.67 centipoise (cP) to about 7.47 cP at about 20° C. However, pH and viscosity data stated for these examples is not meant as limiting.

### Additional Samples

### Sample Formulations with Antioxidants and Other Health-Promoting Compounds

TABLE 3
Alternative embodiments prepared from Examples 1-6, by
adding at least one other health-promoting compound.

| Compound | Quantity (/10 mL) | |
|---|---|---|
| Oil soluble vitamins: | | |
| vitamin E | 50 | mg |
| vitamin D | 5 | micrograms |
| vitamin A | 900 | micrograms |
| Water soluble vitamins: | | |
| vitamin B₁ | 1.2 | mg |
| vitamin B₂ | 1.3 | mg |
| vitamin C | 100 | mg |

In the samples described in Table 3, the oil soluble vitamins were added to the oil phase. The water soluble vitamins were added to the water phase. Stirring and dispersing were performed as described and taught herein. The addition of these other health-promoting compounds did not significantly affect pH or viscosity beyond stated ranges.

### Additional Samples—High Viscosity Formulations

The inclusion of 160 mg (in a 10 mL batch) of whey protein as a health-promoting compound did not significantly affect pH beyond stated ranges. However, viscosity was significantly higher. The oil phase to which the whey protein was added was stirred overnight to achieve clear solution. Dispersing of the oil phase into the water phase for samples containing whey protein underwent sonication for at least about one hour.

The inclusion of 100 mg (in a 10 ml batch) of the antioxidant, squalene, did not significantly affect pH beyond stated ranges. The water phase to which the squalene was added was stirred overnight to achieve clear solution. Compared to the nanoemulsions containing other antioxidants, viscosity of the nanoemulsions containing squalene was significantly higher, ranging from about 13 cP to about 15 cP. At temperatures of 5° C. and 25° C., respectively, all sample formulations remained stable for at least twelve months without significant changes in physical properties.

### Example 7

### Malonaldehyde (MDA) Production in Mice Following Oral Administration

Male adult mice were obtained having either the human APOE ε4 allele (targeted replacement, n=42) or wild type allele (n=42). As shown in Table 4, below, 28 of each kind received oral administration of either vitamin E (Example 2, 5 mg/65 kg body wt) or lipoic acid (Example 3, 100 mg/65 kg body wt), either dissolved in sterile water (SW) or in nanoemulsified form. Administration was by gavage once a day for 120 days. After this period, the subject mice underwent sleep fragmentation (SF) for 15 days. The SF device exposes freely behaving mice in a standard laboratory cage to intermittent tactile stimulation utilizing a motorized mechanical device. Tactile stimulation is produced by a horizontal bar powered by an electrical motor system, which sweeps just above the cage floor from side to side at intermittent functioning mode (2 min). During the sweeper motion, mice may need to step over the sweeper as it passes.

Chronic SF may induce inflammatory responses and oxidative stress. After 15 days of SF, the mice were sacrificed and the brain dissected out for MDA and 8-OHdG assays, which are produced by the brain in response to oxidative stress and inflammatory events.

MDA production is an indicator of lipid peroxidation. After the mice were sacrificed, an LPO-586 kit (OxisResearch, Portland Oreg.) was used according to the manufacturer's instructions to measure MDA levels in the brain tissue. The animals were anesthetized with CO₂ inhalation then decapitated, and the cortex were dissected, snap frozen in liquid nitrogen, and stored at −80° C. until assay the following day. Neuronal tissues were homogenized in 20 mM phosphate buffer (pH 7.4) containing 0.5 mM butylated hydroxytoluene to prevent sample oxidation. Protein concentrations were determined by the Lowry protein assay, and equal amounts of proteins (2.0 to 2.5 mg proteins from each sample) in triplicates were reacted with chromogenic reagents at 45° C. in 500 μl buffer for 1-2 hours. The samples were then centrifuged and clear supernatants measured at 586 nm. MDA production was then calculated per the standard curve according to the manufacturer's instructions. The relative levels of lipid peroxidation were expressed in fold increase in MDA production. The results showed a decrease in MDA, as seen in the following FIG. 1 and Table 4.

TABLE 4
Comparative levels of MDA production following oral
administration of antioxidants.
MDA-Wild Type

| Comparison | Diff of Means | p | q | P |
|---|---|---|---|---|
| lipoic acid (nano) | 0.679 | 7 | 10.387 | <0.001 |
| lipoic acid (SW) | 0.46 | 7 | 7.032 | <0.001 |
| vitamin E (nano) | 0.53 | 7 | 8.111 | <0.001 |
| vitamin E (SW) | 0.489 | 7 | 7.49 | <0.001 |
| control | 0.554 | 7 | 8.475 | <0.001 |
| vehicle | 0.531 | 7 | 8.125 | <0.001 |

MDA-APOE ε4 allele

| Comparison | Diff of Means | p | q | P |
|---|---|---|---|---|
| lipoic acid (nano) | 0.836 | 7 | 5.781 | 0.003 |
| lipoic acid (SW) | 0.663 | 7 | 4.584 | 0.005 |
| vitamin E (nano) | 0.884 | 7 | 6.112 | 0.005 |
| control | 0.774 | 7 | 5.355 | 0.011 |
| vehicle | 0.84 | 7 | 5.809 | 0.041 |

It is to be understood that the embodiments described herein are not limited in their application to the details of the teachings and descriptions set forth herein, or as illustrated in the above examples. Rather, it will be understood that nanoemulsions containing antioxidants and other health-promoting compounds, as taught and described according to multiple embodiments disclosed herein, are capable of other embodiments and of being practiced or of being carried out in various ways.

Also, it is to be understood that the phraseology and terminology used herein is for the purpose of description and should not be regarded as limiting. The use herein of “including,” “comprising,” “e.g.,” “containing,” or “having” and variations of those words is meant to encompass the items listed thereafter, and equivalents of those, as well as additional items.

Accordingly, the foregoing descriptions of several embodiments and alternatives are meant to illustrate, rather than to serve as limits on the scope of what has been disclosed herein. The descriptions herein are not intended to be exhaustive, nor are they meant to limit the understanding of the embodiments to the precise forms disclosed. It will be understood by those having ordinary skill in the art that modifications and variations of these embodiments are reasonably possible in light of the above teachings and descriptions.

We claim:

1. A composition comprising at least one oil; at least one emulsifying agent; at least one antioxidant; and water; wherein the composition comprises particles having diameters no larger than approximately 500 nm, and each particle comprises an outer shell layer containing the at least one oil, and a core portion containing the at least one antioxidant.

2. The composition of claim 1, wherein the diameter of each particle is about 50 nm-350 nm.

3. The composition of claim 1, wherein the diameter of each particle is equal to or less than about 130 nm.

4. The composition of claim 1, wherein the composition is a nanoemulsion for oral administration to a mammalian subject.

5. The composition of claim 4, wherein the subject is a human.

6. The composition of claim 4, wherein the at least one oil is chosen from the group soybean oil, olive oil, peanut oil, safflower oil, canola oil, corn oil, vitamin E, vitamin A, and combinations thereof;
the at least one emulsifying agent is chosen from the group tragacanth, acacia, agar, chondrus, pectin, lecithin, lanolin, cholesterol, methyl cellulose, sodium carboxylmethyl cellulose, hydroxyl propyl cellulose, cetrimide, benzalkonium chloride, benzethonium chloride, glyceryl monostearate, propylene glycol monosteatrate, polyoxyl stearate, bentonite, aluminium magnesium stearate, attapulgite, colloidal anhydrous silica, hectorite, and combinations thereof;
and the at least one antioxidant is chosen from the group vitamin A, vitamin E, vitamin K, beta carotene, curcumin, Coenzyme Q10, ethoxyquin, ginkgo biloba, glutathione, huperzine, lipoic acid, lycopene, melatonin, resveratrol, quercetin, catechin, epicatechin, hydroxytyrosol, 5-hydroxytryptophan, squalene, apolipoprotein E, bilirubin, creatine, uric acid, carotenoids, flavonoids, lignans, and combinations thereof.

7. The composition of claim 4, wherein the at least one oil is lipophilic.

8. The composition of claim 6, further comprising a stabilizing agent chosen from the group ethylene glycol, propylene glycol, polyethylene glycol, glycerin, and combinations thereof.

9. The composition of claim 8, further comprising a preservative chosen from the group methylparaben, propylparaben, sodium metabisulphite, and combinations thereof.

10. The composition of claim 6, further comprising at least one health-promoting compound chosen from the group soy protein, whey protein, vitamin D, vitamin C, vitamin B₁, vitamin B₆, and vitamin B₁₂, and combinations thereof.

11. A method of administering the composition of claim 4 to a subject, wherein the method is chosen from the group oral administration, topical administration, intravenous administration, transdermal administration, and subcutaneous administration.

12. A method of forming a composition for administration to a mammalian subject, which comprises particles that pass across the blood-brain barrier after administration, comprising the steps of preparing an oil phase comprising at least one oil, at least one emulsifying agent, and at least one antioxidant; preparing a water phase comprising water and at least one stabilizing agent; and dispersing the oil phase into the water phase.

13. The method of claim 12, wherein the particles are no larger than approximately 500 nm and comprise an outer shell layer containing the at least one oil, and a core portion containing the at least one antioxidant.

14. The method of claim 12, wherein the diameter of each particle is about 50 nm-350 nm.

15. The method of claim 12, wherein the diameter of each particle is equal to or less than about 130 nm.

16. The method of claim 12, wherein the at least one oil is chosen from the group soybean oil, olive oil, peanut oil, safflower oil, canola oil, corn oil, vitamin E, vitamin A, and combinations thereof;
the at least one emulsifying agent is chosen from the group tragacanth, acacia, agar, chondrus, pectin, lecithin, lanolin, cholesterol, methyl cellulose, sodium carboxyl methyl cellulose, hydroxyl propyl cellulose, cetrimide, benzalkonium chloride, benzethonium chloride, glyceryl monostearate, propylene glycol monosteatrate, polyoxyl stearate, bentonite, aluminium magnesium stearate, attapulgite, colloidal anhydrous silica, hectorite, and combinations thereof the at least one antioxidant is chosen from the group vitamin A, vitamin E, vitamin K, beta carotene, curcumin, Coenzyme Q10, ethoxyquin, ginkgo biloba, glutathione, huperzine, lipoic acid, lycopene, melatonin, resveratrol, quercetin, catechin, epicatechin, hydroxytyrosol, 5-hydroxytryptophan, squalene, apolipoprotein E, bilirubin, creatine, uric acid, carotenoids, flavonoids, lignans, and combinations thereof;
and the at least one stabilizing agent is chosen from the group ethylene glycol, propylene glycol, polyethylene glycol, glycerin, and combinations thereof.

17. The method of claim 16, further comprising the step of adding to the water phase a preservative chosen from the group methylparaben, propylparaben, sodium metabisulphite, and combinations thereof.

18. The method of claim 17, further comprising the step of adding to the water phase an additive chosen from the group squalene and aspartame, and combinations thereof.

19. The method of claim 16, further comprising the step of adding to the oil phase at least one health-promoting compound chosen from the group soy protein, whey protein, vitamin D, vitamin C, vitamin B₁, vitamin B₆, and vitamin B₁₂, and combinations thereof.

20. The method of claim 13, wherein the composition is administered to the subject through a method of administration chosen from the group oral administration, topical administration, intravenous administration, transdermal administration, and subcutaneous administration.
